(19) United States
(12) Patent Application Publication
(10) Pub. No.: US 2025/0121131 A1
(43) Pub. Date: Apr. 17, 2025

(54) MEDICAL DEVICE HUB POWER MANAGEMENT SYSTEM, METHOD, AND APPARATUS

(71) Applicants: BAXTER INTERNATIONAL INC., Deerfield, IL (US); BAXTER HEALTHCARE SA, Glattpark (Opfikon) (CH)

(72) Inventors: Peter BOJAN, Grayslake, IL (US); D. Gnana PRASUNA, Andhra Pradesh (IN); Siba Prasad SAHU, Bangalore (IN); David James SCHMIDT, Gurnee, IL (US); Subbaiah PATIBANDLA, Karnataka Bangalore (IN); Erik Michael THOMAS, Gurnee, IL (US); Niranjan MANJUNATH, Basaweswarnagar Bangalore (IN); Yogesh MUNAVALLI, Karnataka Bangalore (IN); Jiri SLABY, Buffalo Grove, IL (US)

(21) Appl. No.: 18/914,871

(22) Filed: Oct. 14, 2024

Related U.S. Application Data
(60) Provisional application No. 63/543,975, filed on Oct. 13, 2023.

(51) Int. Cl. A61M 5/145

(52) U.S. Cl. CPC A61M 5/1452; A61M 2205/3561; A61M 2205/50; A61M 2209/086

(57) ABSTRACT

A medical device hub power management system, method, and apparatus are disclosed. An example hub connectivity station includes a connectivity stage configured to provide a data connection with an external network. The hub connectivity station also includes at least one docking apparatus configured to connect to at least one medical device. The docking apparatuses are connected to the connectivity station in a stacked arrangement. The docking apparatuses and connectivity stage include power and data connectors to enable data and power to be provided through the hub connectivity station. To prevent electrical shock, a top power connector of each docking apparatus is disconnected from power when that docking apparatus is not connected to another docking apparatus or the connectivity stage. Power is provided to the top power connector after detecting another docking apparatus or the connectivity stage is connected to the top of the docking apparatus.

### PRIORITY CLAIM

This application claims priority to and the benefit of U.S. Provisional Patent Application Ser. No. 63/543,975, filed Oct. 13, 2023, the entire contents of which are hereby incorporated by reference and relied upon.

### BACKGROUND

Infusion pumps are medical devices that infuse a fluid into a patient's bloodstream or subcutaneously. Infusion therapies often use pumps to delivery nutrition in addition to certain medications such as pain medications, antibiotics, antiemetics, antifungals, antivirals, biologics, blood factors, chemotherapy drugs, corticosteroids, growth hormones, immunoglobulin replacement fluids, immunotherapy fluids, and inotropic heart medications. There are different types of infusion pumps that are optimized for delivering different types of fluids or medications. For instance, a syringe pump may be used for delivering low volumes of potent medications at relatively slow rates while large volume parenteral (“LVP”) pumps are used for delivering greater volumes of nutritional fluids. Other types of known infusion pumps include linear peristaltic pumps, patient-controlled analgesia (“PCA”) pumps, ambulatory pumps, and multi-channel pumps.

Some patient treatments may require the use of more than one infusion pump. For example, a first infusion pump may deliver saline while a second pump delivers a medication. Currently, clinicians can either connect individual pumps to a patient or use a multi-channel pump. When individual pumps are used, the pumps are often spread around a patient's hospital bed. The clinician has to move around the patient's bed to program each pump individually. In addition, the pumps consume a significant amount of space. When a multi-channel pump is used, a clinician only has to program one device, and less space is used. However, multi-channel pumps generally only support one type of infusion pump, such as large volume pumps. If another pump is needed for a treatment, a clinician still has to add that infusion pump separately from and in addition to the multi-channel pump, thereby reducing the efficiencies of the multi-channel pump.

A need accordingly exists for an infusion pump hub that supports different types of infusion pumps.

### SUMMARY

An example system, method, and apparatus are disclosed for power management of a hub connectivity station for medical devices, such as infusion pumps. Unlike known multi-channel infusion pump systems with a fixed two, four, or six pumps, the hub connectivity station described herein is modular. In a base configuration, the hub connectivity station includes a single medical device (infusion pump) docking apparatus and a connectivity stage. As disclosed herein, the connectivity stage connects the hub to a hospital network. The docking apparatus includes two shelves for respectively receiving infusion pumps or other medical devices. In an embodiment, the shelves are configured to interchangeably accommodate different types of infusion pumps, such as syringe pumps, LVP pumps, PCA pumps, etc. based on which type of pump is needed for a particular treatment. Further, depending on the number of infusion pumps needed, additional docking apparatuses may be added in a stacked configuration. The hub connectivity station accordingly provides a compact and adaptable infusion management system that requires a relatively small footprint.

The example system, method, and apparatus are configured to distribute power and data among one or more docking apparatuses and the connectivity stage. As disclosed herein, the connectivity stage is positioned at a top of the hub connectivity station. A top of a first docking apparatus is connected to a bottom of the connectivity stage. A top of a second docking apparatus may then be connected to a bottom of the first docking apparatus. Additional docking apparatuses are added in a similar manner such that the hub connectivity station consists of one connectivity stage and one or more stacked docking apparatuses. A power cord is connected to the bottom docking apparatus to provide power from a wall outlet or other power source. As such, only a single power cord is needed to power all of the medical devices connected to the hub connectivity station disclosed herein.

To enable the AC voltage from the wall outlet to be routed to higher-stacked docking apparatuses and the connectivity stage, each docking apparatus includes a power bus and power connectors that are configured to mate with corresponding power connectors of sequentially stacked docking apparatuses. Since the connectivity stage is located at a top of the hub connectivity station, only the bottom of the connectivity stage includes a power connector. Each docking apparatus and the connectivity stage include their own voltage regulator to convert the AC voltage from the power bus to one or more DC voltages. This electrical configuration enables docking apparatuses to be connected in a series configuration without over burdening a single voltage regulator when new docking apparatuses and corresponding infusion pumps are added to the hub connectivity station.

The top power connector of each docking apparatus poses a potential safety risk when a bottom docking apparatus is powered. When such a docking apparatus is powered and not connected to a higher docking apparatus or a connectivity stage (or when the higher docking apparatus or connectivity stage is removed), the top power connector of that lower docking apparatus is exposed. Contact with the power connector could shock a clinician or create a short-circuit. To eliminate this potential safety issue, each docking apparatus includes a relay switch that is configured to be in a closed positioned only when a detection circuit detects that a higher docking apparatus (or connectivity stage) is connected. Absent a detection of a higher docking apparatus, the relay switch is configured to be in an open state or position, thereby preventing the AC voltage from reaching the top power connector of the docking apparatus. The relay switch and the detection circuit accordingly prevent the top power connector from providing a shock or short-circuit when a higher docking apparatus or a connectivity stage is not connected.

As discussed herein, the detection circuit is configured to accurately detect when a higher docking apparatus is connected to a lower docking apparatus. Instead of a direct mechanical switch that completes a circuit, the detection circuit includes a pin to which a lower voltage or ground is applied only when a corresponding connector of the higher docking apparatus is mated for at least a threshold amount of time. This configuration prevents inadvertent contact with the lower docking apparatus from accidently triggering the relay switch.

Aspects of the subject matter described herein may be useful alone or in combination with one or more other aspect described herein. Without limiting the foregoing description, in a first aspect of the present disclosure, an infusion pump docking apparatus of a hub connectivity station includes a housing, an AC inlet connector positioned on a bottom side of the housing, and an AC outlet connector positioned on a top side of the housing and aligned with and electrically coupled to the AC inlet connector. The docking apparatus also includes a relay switch circuit electrically connected between the AC outlet connector and the AC inlet connector, a voltage regulator configured to convert an AC voltage received from the AC inlet connector to at least one DC voltage, and a microcontroller configured to receive at least one DC voltage from the voltage regulator. The docking apparatus further includes a first data connector positioned on the bottom side of the housing and a second data connector positioned on the top side of the housing and aligned with the first data connector. Additionally, the docking apparatus includes a detection circuit electrically connected between the microcontroller and the second data connector. The detection circuit is configured to detect when a first data connector of another infusion pump docking apparatus is coupled to the infusion pump docking apparatus. The detection circuit is configured to provide a signal to the microcontroller indicative of the connection of the other infusion pump docking apparatus. The microcontroller is configured to cause the relay switch circuit to close to enable the AC voltage to reach the AC outlet connector for powering the other infusion pump docking apparatus when connected to the infusion pump docking apparatus.

In accordance with a second aspect of the present disclosure, which may be used in combination with the first aspect, the apparatus further includes a first pump connector configured to provide at least one DC voltage from the voltage regulator to a first infusion pump, a first shelf and latch to mechanically connect to and support the first infusion pump, a second pump connector configured to provide at least one DC voltage from the voltage regulator to a second infusion pump, and a second shelf and latch to mechanically connect to and support the second infusion pump.

In accordance with a third aspect of the present disclosure, which may be used in combination with any other aspect disclosed herein, the other infusion pump docking apparatus is identical to the infusion pump docking apparatus.

In accordance with a fourth aspect of the present disclosure, which may be used in combination with any other aspect disclosed herein, the other infusion pump docking apparatus is a top-stage connectivity stage that omits an AC outlet connector, a relay switch circuit, a second data connector, and a detection circuit.

In accordance with a fifth aspect of the present disclosure, which may be used in combination with the first aspect, a pin of the first data connector is electrically coupled to ground or a low voltage, a pin of the second data connector is configured to electrically connect to the pin of the first data connector of the other infusion pump docking apparatus when connected, and the detection circuit is configured to detect the connection of the other infusion pump docking apparatus when the pin of the second data connector is pulled to the ground or the low voltage.

In accordance with a sixth aspect of the present disclosure, which may be used in combination with any other aspect disclosed herein, each of the first data connector and the second connector additionally includes pins for at least one of controller area network (“CAN”) communication, Ethernet communication, and universal asynchronous receiver-transmitter (“UART”) communication.

In accordance with a seventh aspect of the present disclosure, which may be used in combination with any other aspect disclosed herein, the detection circuit includes an inverter with a Schmitt trigger for detecting when the pin of the second data connector is pulled to the ground or the low voltage, an output of the inverter being electrically connected to the microcontroller.

In accordance with an eighth aspect of the present disclosure, which may be used in combination with any other aspect disclosed herein, the relay switch circuit includes a relay switch, a logic circuit including a first input that is connected to the output from the inverter, a second input that is connected to an activation signal pin of the microcontroller, and an output for providing a relay activation signal, and a relay driver configured to receive the relay activation signal and provide an excitation voltage to the relay switch, causing the relay switch or actuate to a closed position.

In accordance with a ninth aspect of the present disclosure, which may be used in combination with any other aspect disclosed herein, the microcontroller includes instructions stored in a memory that define an algorithm that when executed, cause the microcontroller to receive an output signal from the inverter, detect the connection of the other infusion pump docking apparatus by comparing the output signal to a threshold, and transmit an activation signal via an activation signal pin when the output signal exceeds the threshold.

In accordance with a tenth aspect of the present disclosure, which may be used in combination with any other aspect disclosed herein, the threshold includes at least one of a voltage amplitude value and a time value.

In accordance with an eleventh aspect of the present disclosure, which may be used in combination with any other aspect disclosed herein, the detection circuit is included within a same circuit packaging as the microcontroller or integrally formed with the microcontroller.

In accordance with a twelfth aspect of the present disclosure, which may be used in combination with any other aspect disclosed herein, an infusion pump docking apparatus of a hub connectivity station includes an AC inlet connector positioned on a bottom side of the housing, an AC outlet connector positioned on a top side of the housing and aligned with and electrically coupled to the AC inlet connector, a relay switch circuit electrically connected between the AC outlet connector and the AC inlet connector, a first data connector positioned on the bottom side of the housing, a second data connector positioned on the top side of the housing and aligned with the first data connector, and a detection circuit electrically connected to the second data connector. The detection circuit is configured to detect when a first data connector of another infusion pump docking apparatus is coupled to the infusion pump docking apparatus and provide a signal indicative of the connection of the other infusion pump docking apparatus. The docking apparatus also includes a microcontroller electrically connected to the detection circuit. The microcontroller is configured to receive the signal from the detection circuit, and cause the relay switch circuit to close to enable the AC voltage to reach the AC outlet connector for powering the other infusion pump docking apparatus when connected to the infusion pump docking apparatus.

In accordance with a thirteenth aspect of the present disclosure, which may be used in combination with any other aspect disclosed herein, the other infusion pump docking apparatus is identical to the infusion pump docking apparatus.

In accordance with a fourteenth aspect of the present disclosure, which may be used in combination with any other aspect disclosed herein, the other infusion pump docking apparatus is a top-stage connectivity stage that omits an AC outlet connector, a relay switch circuit, a second data connector, and a detection circuit.

In accordance with a fifteenth aspect of the present disclosure, which may be used in combination with any other aspect disclosed herein, a pin of the first data connector is electrically coupled to ground or a low voltage, a pin of the second data connector is configured to electrically connect to the pin of the first data connector of the other infusion pump docking apparatus when connected, and the detection circuit is configured to detect the connection of the other infusion pump docking apparatus when the pin of the second data connector is pulled to the ground or the low voltage.

In accordance with a sixteenth aspect of the present disclosure, which may be used in combination with any other aspect disclosed herein, each of the first data connector and the second connector additionally includes pins for at least one of controller area network (“CAN”) communication, Ethernet communication, and universal asynchronous receiver-transmitter (“UART”) communication.

In accordance with a seventeenth aspect of the present disclosure, which may be used in combination with any other aspect disclosed herein, the detection circuit includes an inverter with a Schmitt trigger for detecting when the pin of the second data connector is pulled to the ground or the low voltage, an output of the inverter being electrically connected to the microcontroller.

In accordance with an eighteenth aspect of the present disclosure, which may be used in combination with any other aspect disclosed herein, the relay switch circuit includes a relay switch, a logic circuit including a first input that is connected to the output from the inverter, a second input that is connected to an activation signal pin of the microcontroller, and an output for providing a relay activation signal, and a relay driver configured to receive the relay activation signal and provide an excitation voltage to the relay switch, causing the relay switch or actuate to a closed position.

In accordance with a nineteenth aspect of the present disclosure, which may be used in combination with any other aspect disclosed herein, the microcontroller includes instructions stored in a memory that define an algorithm that when executed, cause the microcontroller to receive an output signal from the inverter, detect the connection of the other infusion pump docking apparatus by comparing the output signal to a threshold, and transmit an activation signal via an activation signal pin when the output signal exceeds the threshold.

In accordance with a twentieth aspect of the present disclosure, which may be used in combination with any other aspect disclosed herein, the threshold includes at least one of a voltage amplitude value and a time value.

In accordance with a twenty-first aspect of the present disclosure, any of the structure and functionality illustrated and described in connection with FIGS. 1 to 12 may be used in combination with any of the structure and functionality illustrated and described in connection with any of the other of FIGS. 1 to 12 and with any one or more of the preceding aspects.

In light of the present disclosure and the above aspects, it is therefore an advantage of the present disclosure to provide a hub power management system that provides power to a connector only after detecting that a medical device docking apparatus is connected.

It is another advantage of the present disclosure to use a relay switch to prevent an AC voltage from reaching an exposed power connector.

It is a further advantage of the present disclosure to provide a hub connectivity station with an AC power connection throughout regardless of a number of docking apparatuses connected.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a perspective view of a hub connectivity station with a single docking apparatus and a connectivity stage, according to an example embodiment of the present disclosure.

FIG. 2 is a diagram of an assembly view of the hub connectivity station of FIG. 1, according to an example embodiment of the present disclosure.

FIG. 3 shows a bottom view of the hub connectivity station of FIG. 1, according to an example embodiment of the present disclosure.

FIG. 4 is a diagram of a hub connectivity station with six docking apparatuses, according to an example embodiment of the present disclosure.

FIG. 5 is a diagram of the hub connectivity station of FIGS. 1 to 3 with two medical devices connected to the docking apparatus, according to an example embodiment of the present disclosure.

FIG. 6 is a diagram of the hub connectivity station communicatively coupled to a gateway server via a network, according to an example embodiment of the present disclosure.

FIG. 7 is a diagram that is illustrative of power routing within the docking apparatus of the hub connectivity station, according to an example embodiment of the present disclosure.

FIG. 8 is a diagram illustrative of power management provided by the docking apparatus, according to an example embodiment of the present disclosure.

FIG. 9 is a diagram of a detection circuit of the docking apparatus, according to an example embodiment of the present disclosure.

FIG. 10 is a diagram of a relay driver of the docking apparatus, according to an example embodiment of the present disclosure.

FIG. 11 is a diagram of the connectivity stage, according to an example embodiment of the present disclosure.

FIG. 12 shows a flow diagram illustrating an example procedure for controlling power between the docking apparatuses and the connectivity stage of the hub connectivity station, according to an example embodiment of the present disclosure.

### DETAILED DESCRIPTION

The present disclosure relates in general to a method, system, and apparatus for power management of a hub connectivity station. As disclosed herein, the hub connectivity station comprises a number of stages that are linked together in a stacked configuration. Each hub connectivity station includes a single connectivity stage that is communicatively coupled to a medical network. Additionally, each hub connectivity station includes one or more medical device docking apparatuses. Each docking apparatus can accommodate, in some embodiments, two medical devices, such as infusion pumps. The number of docking apparatuses used in the hub connectivity station depends on the number of medical devices needed for a patient treatment. Together, the connectivity stage and the docking apparatuses provide communication and power routing.

In contrast to the hub connectivity station disclosed herein, known multi-channel infusion pumps include a single controller and two to six pumps. The controller provides centralized management of the infusion pumps such that the pumps cannot be removed during operation. Additionally, the controller is configured to operate with only one type of infusion pump, thereby limiting system flexibility.

The hub connectivity station overcomes at least some of the limitations of known multi-channel infusion pumps by enabling multiple docking apparatus to be stacked, where each docking apparatus can accommodate one of many different types of medical devices. Additionally, the medical devices are configured to operate independently and may be removed from the hub connectivity station as needed, even during a treatment without interruption. Such a configuration provides a scalable, flexible, and adaptable system that concentrates medical devices into a relatively small footprint.

### Hub Connectivity Station Embodiment

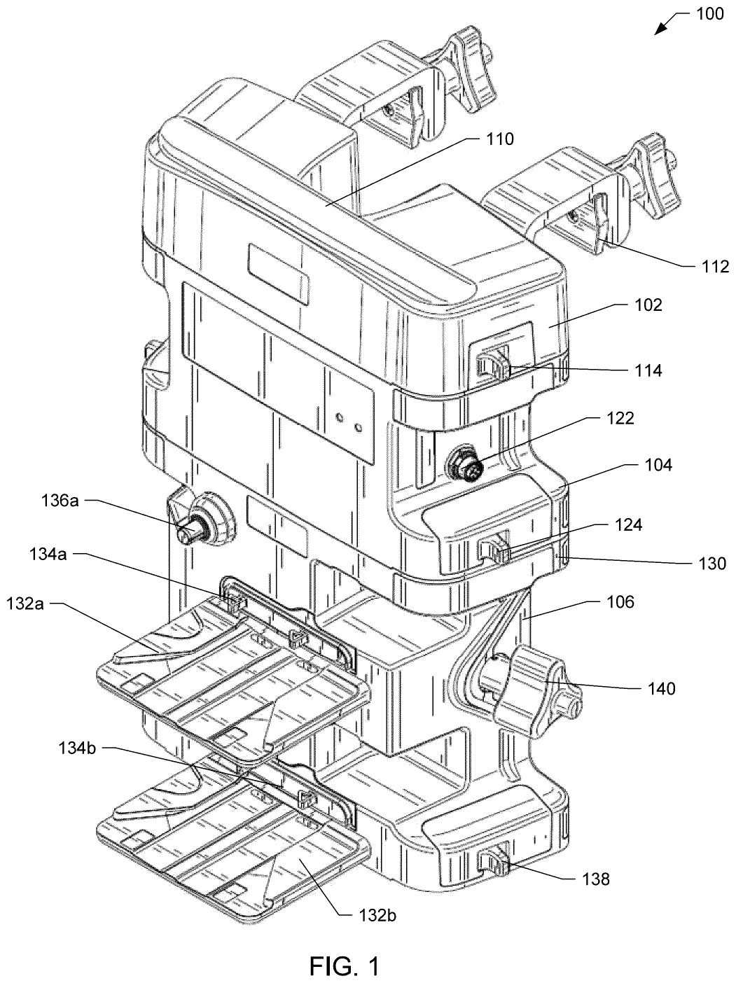

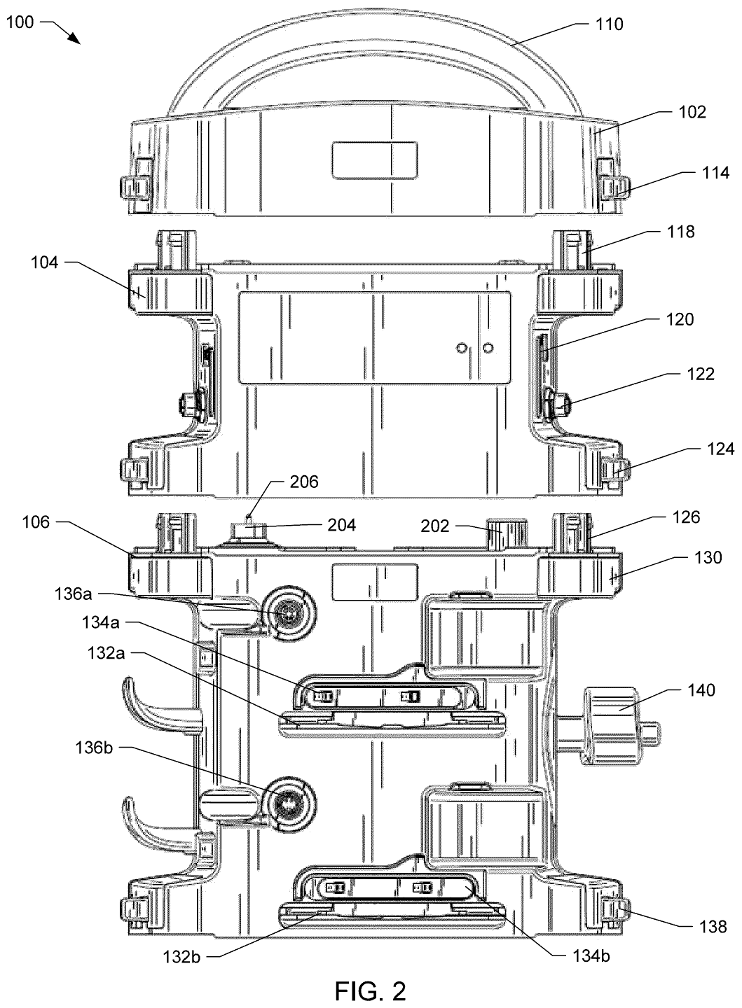

FIGS. 1 and 2 are diagrams of a hub connectivity station 100, according to an example embodiment of the present disclosure. FIG. 1 shows the hub connectivity station 100 from a perspective view. FIG. 2 shows the hub connectivity station 100 from a front assembly view.

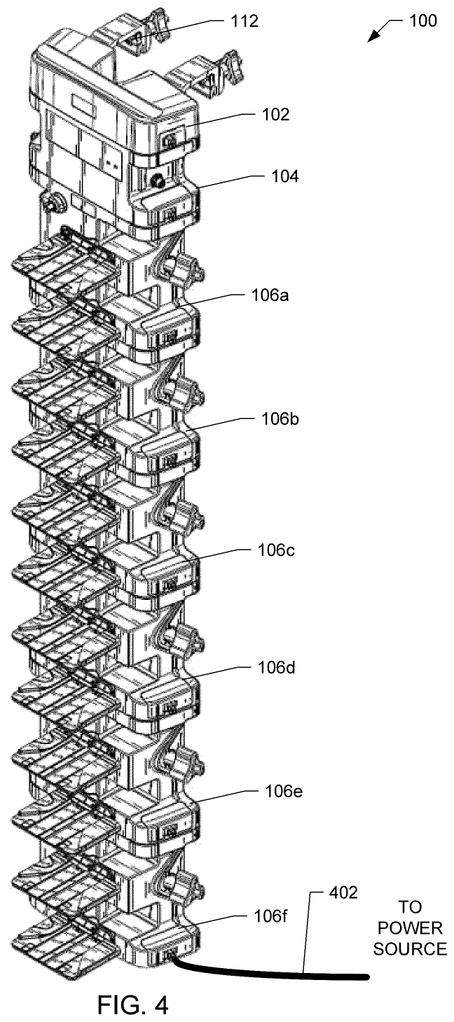

The hub connectivity station 100 includes a handle stage 102, a connectivity stage 104, and a medical device docking apparatus 106. While only one medical device docking apparatus 106 is shown in FIGS. 1 and 2, the hub connectivity station 100 can include additional medical device docking apparatuses 106, as shown in FIG. 4. In some embodiments, the handle stage 102 may be omitted.

As shown in FIGS. 1 and 2, the handle stage 102 includes a handle 110, a bedrail clamp 112, and a release lever 114. The handle 110 is configured to enable the hub connectivity station 100 to be carried by a clinician. The example handle 110 has a semi-circular shape that extends vertically from a housing of the handle stage 102. The handle 110 may be connected to or integrally formed with the housing of the handle stage 102 to support the weight of the connectivity stage 104 and the one or more docking apparatuses 106 during transportation to other locations within a hospital or other medical environment.

The bedrail clamp 112 includes a bracket and a release knob to enable the hub connectivity station 100 to be connected to railing or panel of a patient's bed. The release knob is rotated in one direction to cause a screw or other actuator to move to a closed position, thereby tightening against a rail or hospital bed panel. The release knob is rotated in an opposite direction to cause the screw or other actuator to move to a closed position. In some embodiments, the bedrail clamp 112 may be omitted.

The release lever 114 is configured, when actuated by a clinician, to enable the handle stage 102 to separate from the connectivity stage 104. As shown in FIG. 2, the connectivity stage 104 includes a protrusion section 118 that is configured to mate with a recess section within the handle stage 102. The protrusion section 118 may include a tab or other mechanical connector that engages the corresponding release lever 114 and slots or protrusions within the recess section of the handle stage 102. Insertion of the protrusion section 118 into the recess section may create a secure connection between the handle stage 102 and the connectivity stage 104 when the release lever 114 is engaged. However, when the release lever 114 is actuated, the protrusion section 118 may be slide from the recess section of the handle stage 102, thereby enabling separation of the connectivity stage 104. While FIGS. 1 and 2 show the release lever 114 located on both sides of the handle stage 102, in other embodiments the release lever 114 is located only on one side of the handle stage 102.

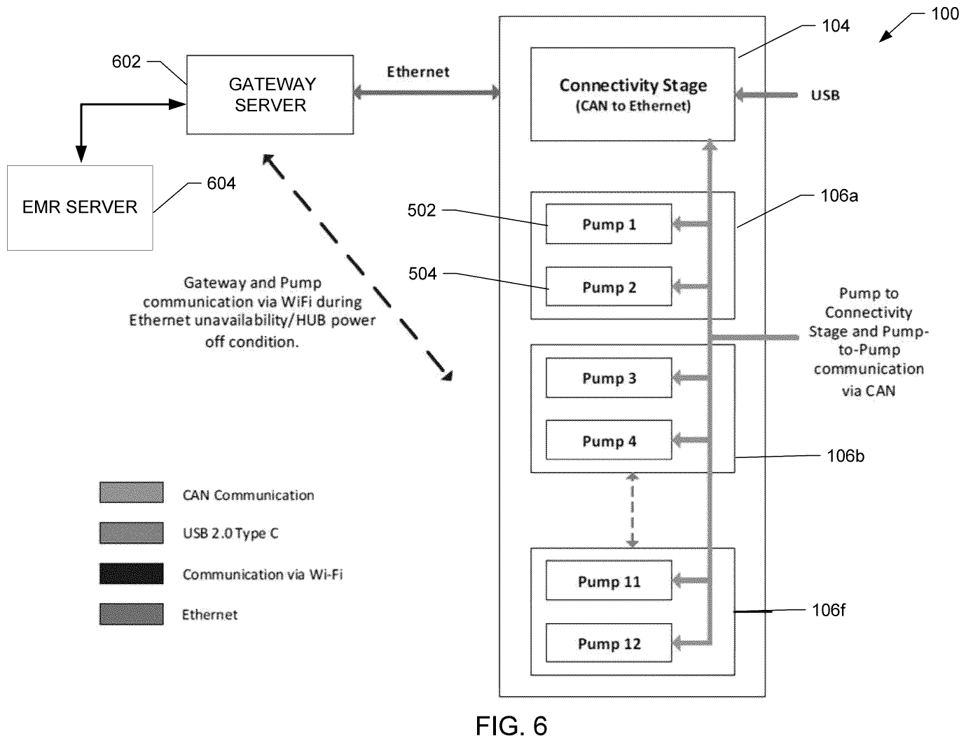

The connectivity stage 104 is configured to provide communication between the hub connectivity station 100 and a hospital network. The communication configuration of the connectivity stage 104 is discussed in more detail in conjunction with FIG. 6. A housing of the connectivity stage 104 includes an Ethernet port 120 (shown in FIG. 2), such as an RJ45 port. The connectivity stage 104 also includes one or more M12 connectors 122. In some embodiments, the M12 connector 122 is omitted. Similar to the handle stage 102, the connectivity stage 104 also includes a release lever 124 to enable the docking apparatus 106 to be removably connected via similar protrusion sections 126.

The docking apparatus 106 is configured to be connected to a bottom of the housing of the connectivity stage 104, as shown in FIGS. 1 and 2. In the illustrated example, the docking apparatus 106 is configured to connect to two medical devices, such as infusion pumps. In other embodiments, the docking apparatus 106 may include a single shelf and connector to couple to only one medical device or have as many as six shelves and six connectors to couple to six medical devices.

The docking apparatus 106 includes a housing 130 and shelves 132a, 132b that extend from the housing 130. Latches 134a, 134b are respectively positioned above each of the shelves 132a, 132b. The latches 134a, 134b are configured to releasably couple to respective medical devices, such as infusion pumps. The shelves 132a, 132b are configured to support a weight of a respective medical device to ensure the medical device does not break away from the respective latch 134a, 134b. The width of the shelves 132a, 132b is configured to correspond to a width of a largest medical device that is to be connected to the docking apparatus 106 such that the largest medical device does not have a significant portion that extends beyond the width of the shelves 132a, 132b. The shelves have a length that is between 50% to 75% of a length of the docking apparatus 106 to provide sufficient support for a connected medical device. Additionally, as shown in FIGS. 1 and 2, the shelves 132a, 132b include recessed sections to guide the placement of a medical device into the respective latch 134a, 134b.

The docking apparatus 106 also includes two device connectors 136a, 136b that extend from the housing 130. The device connectors 136a, 136b are located on the housing 130 of the docking apparatus 106 so as to mate with respective connectors on compatible medical devices. In the illustrated example, the device connectors 136a, 136b have a cylindrical shape that protrudes from the housing 130. In other examples, the device connectors 136a, 136b may be ports that are recessed into the housing 130 and/or have a rectangular or hexagonal shape. Further, while the device connectors 136a are shown as being located above the respective latches 134a, 134b, in other embodiments, they may be offset from the latches 134a, 134b.

The device connectors 136a, 136b are configured to include pins or other contacts for power and data routing between a medical device and the docking apparatus 106. In an example, the device connectors 136a, 136b include a device-present pin for detecting the connection of a medical device. Circuitry within the medical device may apply a low voltage or ground to the device-present pin when the medical device is connected, for example, to the device connector 136a. The docking apparatus 106 is configured to detect when the device-present pin receives a low-voltage or ground to determine when the device is connected.

The device connectors 136a, 136b may also include pins for a controller area network (“CAN”) connection (e.g., a CAN high signal pin and a CAN low signal pin) and/or an Ethernet connection (e.g., positive and negative transmission and receiving signal pins). In some embodiments, the device connectors 136a, 136b may include an identifier request pin. In these embodiments, the identifier request pin is used by the docking apparatus 106 and/or the connectivity stage 104 to transmit a signal or a message requesting that the medical device provide a CAN identifier (e.g., a CAN identification Query ID) through the CAN connection.

FIGS. 1 and 2 also show the housing 130 of the docking apparatus 106 includes a release lever 138, which is similar to the release lever 124 of the connectivity stage 104. Similar to the respective connections between the handle stage 102 and the connectivity stage 104 and the connectivity stage 104 and the docking apparatus 106, the docking apparatuses 106 may be removably connected to each other in a stacked arrangement. The release lever 138 enables the docking apparatus 106 to be disconnected from a lower-positioned docking apparatus.

As discussed above, the hub connectivity station 100 is configured to route power and data through the stack of the connectivity stage 104 and one or more docking apparatuses 106. FIG. 2 shows connectors that are positioned at a top of the housing 130 of the docking apparatus 106. The connectors include an AC outlet connector 202 and a top data connector 204. The AC outlet connector 202 is configured to protrude from a top of the housing 130 and may include an International Electrotechnical Commission (“IEC”) connector with countersunk holes. The connector 202 may have 10 ampere, 250 AC voltage rating.

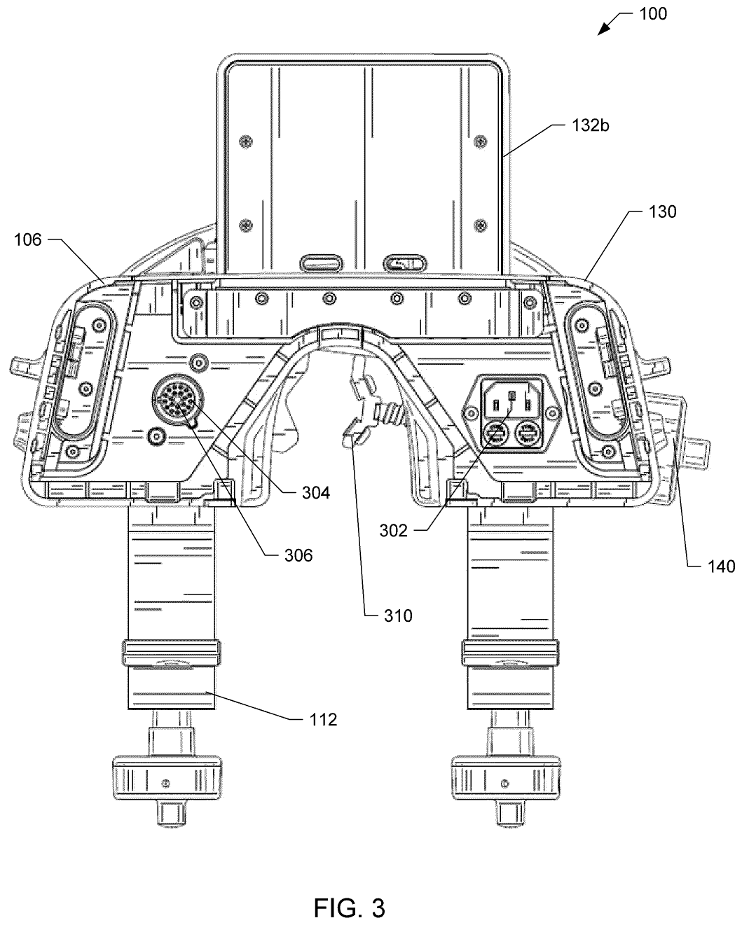

The AC outlet connector 202 is configured to connect to a corresponding AC inlet connector that is provided at a bottom of the connectivity stage 104 and other docking apparatuses 106. FIG. 3 shows a bottom view of the docking apparatus 106, according to an example embodiment of the present disclosure. It should be appreciated that the bottom view is similar for the connectivity stage 104. As shown, a bottom of the housing 130 of the docking apparatus 106 includes an AC inlet connector 302 that is aligned with the AC outlet connector 202 at the top of the housing. The AC inlet connector 302 may include a panel mounted appliance inlet that sits within a recess section of the docking apparatus 106. When connected, the prongs of the AC inlet connector 302 slide into the apertures of the AC outlet connector 202 as a body of the AC outlet connector 202 fits within a recess of the AC inlet connector 302.

The aligned positioning of the AC inlet connector 302 and the AC outlet connector 202 enable the connectivity stage 104 and the docking apparatuses 106 to be electrically connected together when stacked within the hub connectivity station 100. Further, the consistent positioning of the AC inlet connector 302 and the AC outlet connector 202 enable any docking apparatus 106 to be connected to any other docking apparatus 106 or the connectivity stage 104. In some alternative embodiments, the AC inlet connector 302 may be positioned on the top of the housing 130 while the AC outlet connector 202 is positioned on the bottom of the housing 130 of the docking apparatus 106. Further, while the connectors 202 and 302 are shown as Type-B connectors for use in North America, in other examples the connectors may be of Type C through Type 0 to enable the hub connectivity station 100 to be used in other parts of the world.

To power the hub connectivity station 100, a power cord is connected to the AC inlet connector 302 at the bottom of a lower-most docking apparatus 106. An opposite end of the power cord may be connected to an electrical outlet, a power rail, a battery, a generator, or any other power source. Such a configuration ensures that the power cord is placed as close to the ground as possible to reduce the number of wires and lines at higher sections of the station 100. Further, the routing of power through the hub connectivity station 100 means that only a single power cord is needed.

In addition to routing power throughout, the hub connectivity station 100 is also configured to enable data to be communicated among the connectivity stage 104 and the docking apparatuses 106. FIG. 2 shows the top data connector 204 adjacent to the AC outlet connector 202 at the top of the housing 130 of the docking apparatus 106. FIG. 3 shows a bottom data connector 304 that is located at the bottom of the housing 130 adjacent to the AC inlet connector 302. The top data connector 204 is configured to connect to a bottom data connector 304 of the connectivity stage 104 or the bottom data connector 304 of another docking apparatus 106.

The data connectors 204, 304 may include pins for CAN communication. In some embodiments, the data connectors 204, 304 may additionally include pins for universal asynchronous receiver-transmitter (“UART”) communication and/or Ethernet communication between the connectivity stage 104 and the stacked docking apparatuses 106 within the hub connectivity station 100. The data connectors 204, 304 each include at least one respective pin 206, 306 to detect when the connectivity stage 104 or another docking apparatus 106 is connected. In some embodiments, the pin 306 of the bottom data connector 304 is electrically coupled to ground or a low voltage. As described in more detail below, a circuit with the docking apparatus 106 is configured to detect when the pin 206 of the top data connector 204 is pulled to the ground or low voltage to detect when another docking apparatus 106 or the connectivity stage 104 is connected.

FIGS. 1 to 3 also show that the docking apparatus 106 includes a pole clamp, which includes a knob 140 and an actuator 310. The pole clamp enables the docking apparatus 106 to securely couple the hub connectivity station 100 to a pole. Since each docking apparatus 106 includes a pole clamp, multiple stacked docking apparatuses 106 are connected to the same pole to securely connect the hub connectivity station 100 when a plurality of medical devices are used. To connect, the knob 140 is rotated, which causes the actuator 310 to engage the pole. To release, the knob 140 is rotated in an opposite direction, which causes the actuator 310 to move away from the pole.

FIG. 4 shows a diagram of the hub connectivity station 100 of FIG. 1 with six docking apparatuses 106a to 106f stacked below the connectivity stage 104, according to an example embodiment of the present disclosure. In the illustrated example, the handle stage 102 is provided at the top of the hub connectivity station 100. The connectivity stage 104 is connected to the bottom of the handle stage 104. A first docking apparatus 106a is connected to a bottom of the connectivity stage 104. A second docking apparatus 106b is connected to a bottom of the first docking apparatus 106a. Further, the third through sixth docking apparatuses 106c to 106f are sequentially stacked. A power cord 402 electrically couples the sixth docking apparatus 106f to a power source, such as a wall outlet.

The illustrated six docking apparatuses 106a to 106f are configured to accommodate twelve medical devices. To support the weight of the docking apparatuses 106a to 106f themselves in addition to the medical devices, each of the docking apparatuses 106a to 106f include pole clamps for connecting to a pole. In the alternative, the bedrail clamp 112 may connect to a bedrail to support the hub connectivity station 100.

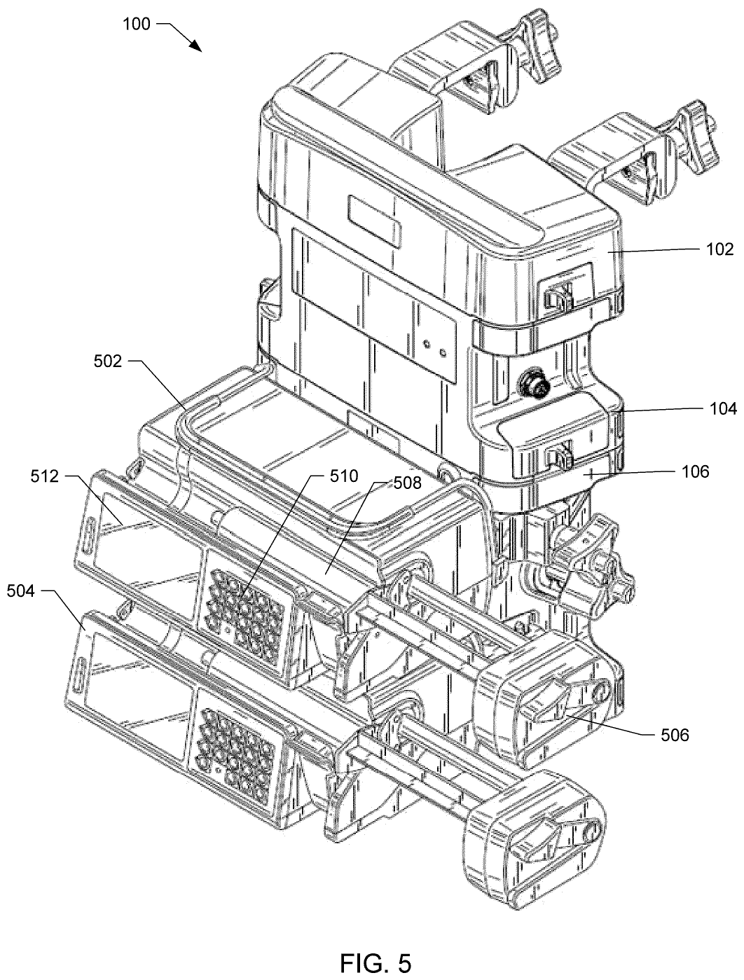

FIG. 5 is a diagram of the hub connectivity station 100 of FIGS. 1 to 3 with two medical devices 502, 504 connected to the docking apparatus 106, according to an example embodiment of the present disclosure. As shown, a first medical device 502 is slide across the shelf 132a to engage the latch 134a. As the medical device 502 is being connected, the device connector 136a of the docking apparatus 106 contacts a corresponding connector of the medical device 502. In a similar manner, a second medical device 504 is slid across the shelf 132b to engage the latch 134b. As the medical device 504 is being connected, the device connector 136b of the docking apparatus 106 contacts a corresponding connector of the medical device 504.

In the illustrated example, the medical devices 502 and 504 are syringe pumps, which include an actuator 506 that presses on a plunger of a syringe 508 to dispense a fluid into an IV tube. The actuator 506 is controlled by a motor within a housing of the medical device 502. Additionally, the medical device 502 includes a user interface comprising a keypad 510 and a display screen 512. The keypad 510 includes one or more buttons switches for controlling operation of the medical device 502. The display screen 512 displays graphics and text regarding the operation of the medical device 502. In some embodiments, the display screen 512 may be a touchscreen. In some embodiments, a clinician uses the keypad 510 and the display screen 512 program an infusion therapy. In addition to manual programming, the medical device 502 may receive electronic prescriptions from a hospital information system via a network. The medical device 502 may include one or more drug libraries that include particular limits based on care area, dose, rate of change, drug type, concentration, patient age, patient weight, etc.

As an infusion pump, the medical device 502 is configured to perform an infusion therapy for a patient, which includes the infusion of one or more fluids, solutions, or drugs into the patient. The medical device 502 operates according to an infusion prescription entered by a clinician at the keypad 510 and/or display screen 512 or received via a network. The medical device 502 may compare the prescription to the drug library and provide any alerts or alarms when a parameter of the prescription violates a soft or hard limit. The medical device 502 is configured to monitor the progress of the therapy and periodically transmit infusion therapy progress data to a gateway server. The infusion therapy progress data may include, for example, an infusion rate, a dose, a total volume infused, a time remaining for the therapy, a fluid concentration, a rate change, a volume remaining within a medication container, a fluid name, a patient identifier, titration information, bolus information, a care area identifier, a timestamp when the data was generated, an alarm condition, an alert condition, an event, etc. In some instances, the infusion therapy progress data includes a new infusion start event including information indicative of an infusion pump identifier, an infused fluid name, an infusion rate, a volume to be infused, a dose, a volume remaining, and/or a time the new infusion start event was generated by the infusion pump. The medical device 502 may transmit the data continuously, periodically (e.g., every 30 seconds, 1 minute, etc.), or upon request by the gateway server. While a syringe pump is shown, the docking apparatus 106 may also connect to other types of infusion pumps such as linear peristaltic pumps, large volume parenteral pumps, ambulatory pumps, PCA pumps, multi-channel pumps, etc.

The medical devices 502 and 504 may also include renal failure therapy (“RFT”) machines, which may include any hemodialysis, hemofiltration, hemodiafiltration, continuous renal replacement therapy (“CRRT”), or peritoneal dialysis machine. CRRT is a dialysis modality typically used to treat critically ill, hospitalized patients in an intensive care unit who develop acute kidney injury (“AKI”). Unlike chronic kidney disease, which occurs slowly over time, AKI often occurs in hospitalized patients and typically occurs over a few hours to a few days. A patient, undergoing hemodialysis, for example, is connected to the RFT machine, where the patient's blood is pumped through the machine. The blood passes through a dialyzer of the machine, which removes waste, toxins and excess water (e.g., ultrafiltrate) from the blood. The cleaned blood is returned to the patient.

Hemodialysis is a renal failure treatment in which waste from the blood is diffused across a semi-permeable membrane. During hemodialysis, blood is removed from the patient and flows through a semi-permeable membrane assembly (dialyzer), where the blood flows generally counter-current to dialysis solution flowing on the other side of the semipermeable membrane. In the dialyzer, toxins from the blood travel across the semi-permeable membrane and exit the dialyzer into used dialysis solution (dialysate). The cleaned blood having flowed through the dialyzer is then returned to the patient.

Hemofiltration is another renal failure treatment, similar to hemodialysis. During hemofiltration, a patient's blood is also passed through a semipermeable membrane (a hemofilter), where fluid (including waste products) is pulled across the semipermeable membrane by a pressure differential. This convective flow brings certain sizes of molecular toxins and electrolytes (which are difficult for hemodialysis to clean) across the semipermeable membrane. During hemofiltration, a replacement fluid is added to the blood to replace fluid volume and electrolytes removed from the blood through the hemofilter. Hemofiltration in which replacement fluid is added to the blood prior to the hemofilter is known as pre-dilution hemofiltration. Hemofiltration in which replacement fluid is added to the blood after the hemofilter is known as post-dilution hemofiltration.

The RFT machine can alternatively be a hemodiafiltration machine.

Hemodiafiltration is a further renal failure treatment that uses hemodialysis in combination with hemofiltration. Blood is again pumped through a dialyzer, which accepts fresh dialysis fluid unlike a hemofilter. With hemodiafiltration, however, replacement fluid is delivered to the blood circuit, like with hemofiltration. Hemodiafiltration is accordingly a neighbor of hemodialysis and hemofiltration.

Alternatively, the medical device 502 may be a peritoneal dialysis machine, which may perform various types of peritoneal dialysis therapies, including continuous cycling peritoneal dialysis (“CCPD”), tidal flow automated peritoneal dialysis (“APD”), and continuous flow peritoneal dialysis (“CFPD”). Peritoneal dialysis infuses dialysate into a patient during fill cycles.

For any dialysis treatment, the RFT machine may compare parameters of a prescription to one or more limits and provide any alerts or alarms when a parameter of the prescription violates a soft or hard limit. The RFT machine is configured to monitor the progress of the therapy and periodically transmit dialysis therapy progress data to a gateway server. The dialysis therapy progress data may include, for example, a fill rate, a dwell time, a drain or fluid removal rate, a blood flow rate, an effluent dose, an ultrafiltration removal rate, a dialysate removal rate, a total dialysate infused, a dialysate flow, a replacement pre-flow, a replacement post-flow, a patient weight balance, a return pressure, an excess patient fluid sign, a filtration fraction, a time remaining, a dialysate concentration, a dialysate name, a patient identifier, a room identifier, a care area identifier, a timestamp when the data was generated, an alarm condition, an alert condition, an event, etc. The RFT machine may transmit the data continuously, periodically (e.g., every 30 seconds, 1 minutes, etc.), or upon request by the gateway server.

In some embodiments, the medical device 502 may include a physiological sensor. For example, the medical device 502 may include a pulse oximetry sensor. Additionally or alternatively, the physiological sensor may include a blood pressure sensor, a patient weight scale, a glucose sensor, a cardiac monitor, etc. In some instances, instead of connecting directly to the docking apparatus 106, the physiological sensor connects to the medical device 502.

In further embodiments, the docking apparatus 106 may also connect to a hemodynamic monitor, which is configured to display information relevant to hemodynamic monitoring and management. This includes fluid balance information, hemodynamic assessment information, hemodynamic parameters, and/or alerts related to infiltration, infusion line occlusions, and/or a fluid bag being near-empty or empty. The hemodynamic monitor uses therapy progress data and/or dialysis therapy progress data in addition to physiological sensor data to determine and/or display hemodynamic information.

### Hub Connectivity Station Communication Routing Embodiment

The example hub connectivity station 100 of FIGS. 1 to 5 is configured to connect to a network. FIG. 6 is a diagram of the hub connectivity station 100 communicatively coupled to a gateway server 602, according to an example embodiment of the present disclosure. The gateway server 602 includes a controller, processor, router, switch, computer, etc. configured to communicate with the hub connectivity station 100 via a network (e.g., a wide area network, a local area network, a wireless local area network, an Ethernet, the Internet, a cellular network, or combinations thereof). The gateway server 602 may be communicatively coupled to more than one hub connectivity station 100. Further, the gateway server 602 may communicate with other medical devices, such as infusion pumps and RFT machines. The gateway server 602 is configured to provide bi-directional communication with the hub connectivity station 100 for the wired/wireless secure transfer of drug libraries, infusion prescriptions, and infusion therapy progress data. The gateway server 602 may also be configured to integrate with a hospital information system to transmit the infusion therapy progress data and/or the dialysis therapy progress data from the medical devices 502, 504 to a hospital electronic medical record (“EMR”) that is managed by an EMR server 604.

The example EMR server 604 is configured to manage patients' EMRs, which are stored in an EMR database. The EMR server 604 receives the infusion therapy progress data and/or the dialysis therapy progress data and uses a machine identifier and/or patient identifier associated with the data to determine a corresponding patient EMR. The EMR server 604 is configured to write the infusion therapy progress data and/or the dialysis therapy progress data to the appropriate patient EMR within the database, thereby providing a record that is accessible to clinicians and medical devices.

As shown in FIG. 6, the connectivity stage 104 is communicatively coupled to the gateway server 602 via a network connection, such as an Ethernet connection. Additionally, the connectivity stage 104 is communicatively coupled to the docking apparatuses 106a to 106f and corresponding medical devices, such as medical devices 502, 504 via a CAN connection. The connectivity stage 104 may associate determine medical device locations within each docking apparatus 106 and an order of the stacked docking apparatuses 106a to 106f using CAN messaging. The connectivity stage 104 then associates CAN identifiers of the medical devices and/or docking apparatuses 106 with Internet Protocol addresses and/or media access control (“MAC”) addresses of the servers 602, 604. This association enables the connectivity stage 104 to convert messages received via the network connection into a CAN message for a specific medical device.

The connectivity stage 104 may also include a USB port, a micro-USB port, an HDMI port, or other hardwire port to enable a medical device, sensor, or computer to be connected. For instance, service technicians may use the USB connection for servicing or installing software updates for the connectivity stage 104 and/or the docking apparatuses 106. In some instances, the USB port may be omitted.

As shown, when the medical devices 502, 504 are connected to the docking apparatus 106a, the medical devices 502, 504 communicate with the gateway server 602 via the connectivity stage 104. When the medical devices 502, 504 are disconnected, they are configured to communicate with the gateway server 602 via a Wi-Fi connection. Further, when the hub connectivity station 100 is powered off (but still connected to a power source), the connectivity stage 104 is deactivated, causing the medical devices 502, 504 to communicate directly with the gateway server 602 via, for example, the Wi-Fi connection. The connectivity stage 104 may include a switch that enables the hub connectivity station 100 to be powered off.

FIG. 6 also shows that the connectivity stage 104 and the docking apparatuses 106a to 106f form a CAN network. After being recognized on the CAN network, the docking apparatuses 106a to 106f may communicate with each other and the connectivity stage 104. Further, connected medical devices 502, 504 may use the CAN network to communicate with each other, the docking apparatuses 106a to 106f, and/or the connectivity stage 104. Such a configuration enables infusion therapy progress data and/or the dialysis therapy progress data to be used for hemodynamic monitoring or managing relayed infusions and other multiple-device dependent treatments.

### Hub Connectivity Station Power Routing Embodiment

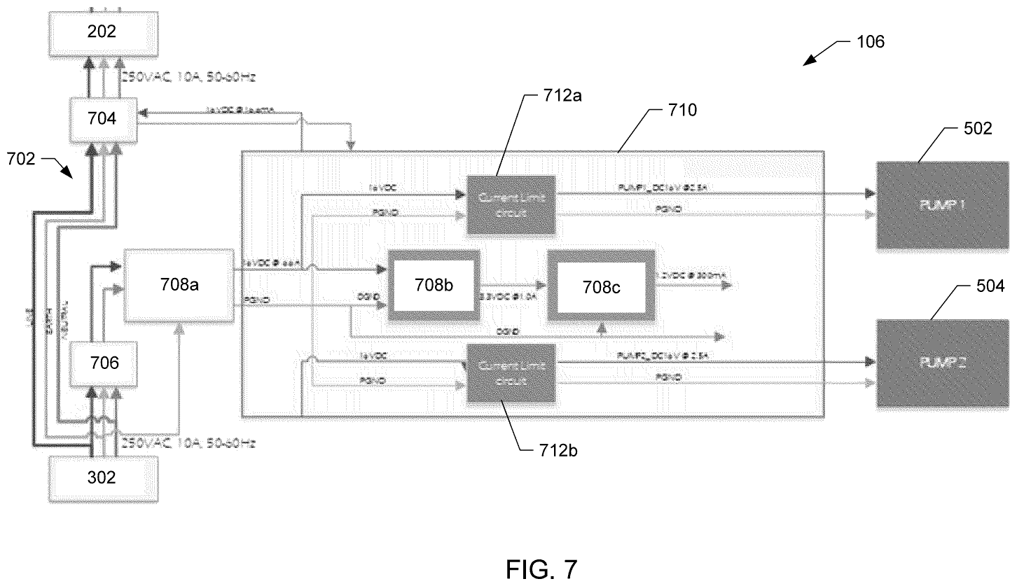

FIG. 7 is a diagram that is illustrative of power routing within the docking apparatus 106 of the hub connectivity station 100, according to an example embodiment of the present disclosure. As disclosed above, the docking apparatus 106 includes the AC outlet connector 202 positioned at the top of the housing 130 and the AC inlet connector 302 positioned at the bottom of the housing 130. Internally, the housing 130 encloses a power bus 702 that electrically couples the AC inlet connector 302 to the AC outlet connector 202. The power bus 702 includes a line wire, an earth wire, and a neutral wire.

Instead of a direct electrical connection between the connectors 202, 302, the docking apparatus 106 includes a relay switch circuit 704. When another docking apparatus or the connectivity stage 104 is connected to the docking apparatus 106 of FIG. 7, the relay switch circuit 704 is configured to be in a closed state, thereby enabling power to reach the AC outlet connector 202. However, when another docking apparatus or the connectivity stage 104 is not connected to the docking apparatus 106, the relay switch circuit 704 is configured to be in an open state, thereby preventing power from reaching the AC outlet connector 202.

To power the docking apparatus 106, the power bus 702 is electrically connected to a filter 706. The example filter 706 may include an AC line filter that is configured to suppress electromagnetic interference (“EMI”) on the power bus 702. In some embodiments, the filter 706 may be omitted. An output of the filter 706 is electrically connected to an AC-DC voltage regulator 708a, which is configured to convert the AC voltage from the power bus 702 to a DC voltage. In the illustrated example, the voltage regulator 708a is configured to output 16 volts. In other embodiments, the voltage regulator 708a may output a lower voltage, such as 5 volts or a greater voltage, such as 24 volts.

In the illustrated example of FIG. 7, the 16 volts from the voltage regulator 708a is further down-converted to 3.3 volts by another voltage regulator 708b. Additionally, the 3.3 volts is down-converted to 1.2 volts by yet another voltage regulator 708c. The additional voltage regulators 708b, 708c may be needed when a microcontroller, processor, or circuit of the docking apparatus 106 requires 3.3 volts and/or 1.2 volts to operate.

The docking apparatus 106 may include at least one printed circuit board (“PCB”) 710 that enables at least the voltage regulators 708b, 708c to be mounted thereto. For power routing, the PCB 710 also includes current limiting circuits 712a, 712b that respectively route the output voltage from the voltage regulator 708a to the medical devices 502, 504. The current limiting circuits 712a, 712b are configured to prevent the medical devices 502, 504 from drawing too much power from the voltage regulator 708a, and the power bus 702 generally. As such, the current limiting circuits 712a, 712b provide short-circuit protection in the event of a failure at one of the medical devices 502, 504.

### Hub Connectivity Station Power Management Embodiment

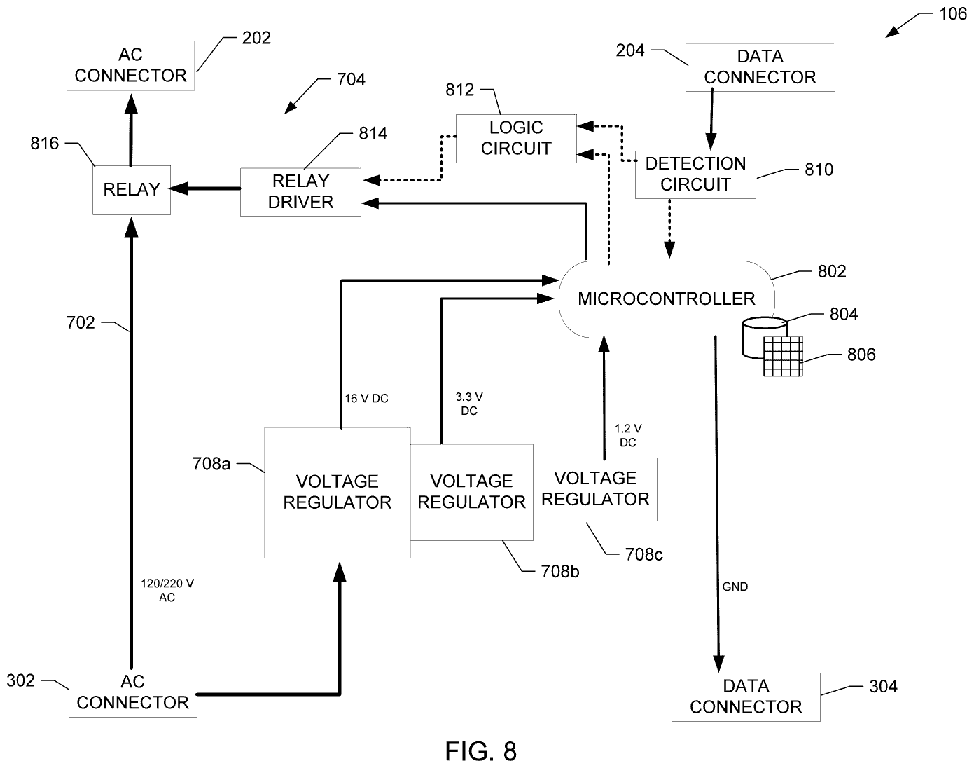

FIG. 8 is a diagram illustrative of the power management provided by the docking apparatus 106, according to an example embodiment of the present disclosure. In the illustrated example, power routing is shown in solid lines while signal and data routing is shown in dashed lines. As discussed in FIG. 7, the docking apparatus 106 includes the AC outlet connector 202, the AC inlet connector 302, the relay switch circuit 704, and the voltage regulators 708a to 708c. Further, as discussed above, the docking apparatus 106 includes the top data connector 204 and the bottom data connector 304.

Additionally, the example docking apparatus 106 includes a microcontroller 802 that receives one or more DC voltages from the voltage regulators 708a to 708c. The microcontroller 802 includes a memory 804 that stores machine-readable instructions 806 that specify operations or an algorithm. Execution of the machine-readable instructions 806 by the microcontroller 802 cause the microcontroller 802 to perform the operations described herein. In some embodiments, the memory 804 may be a separate memory device that is communicatively coupled to the microcontroller 802. Further, in some alternative embodiments, the microcontroller 802 may include a logic controller, a logic implementer, an application specific integrated circuit (“ASIC”), a processor, etc. Further, the memory device 804 may include a flash drive, a solid state drive, or a hard disk drive.

As discussed herein, the microcontroller 802 is configured to operate in conjunction with a detection circuit 810 to control whether the relay switch circuit 704 is actuated to the open state or the closed state. The detection circuit 810 is electrically connected between the microcontroller 802 and the top data connector 204. The example detection circuit 810 is configured to detect when a bottom data connector of another docking apparatus is coupled to the docking apparatus 106 shown in FIG. 8. The detection circuit 810 is configured to transmit a signal (or message) to the microcontroller 802 that is indicative of the connection of the other infusion docking apparatus.

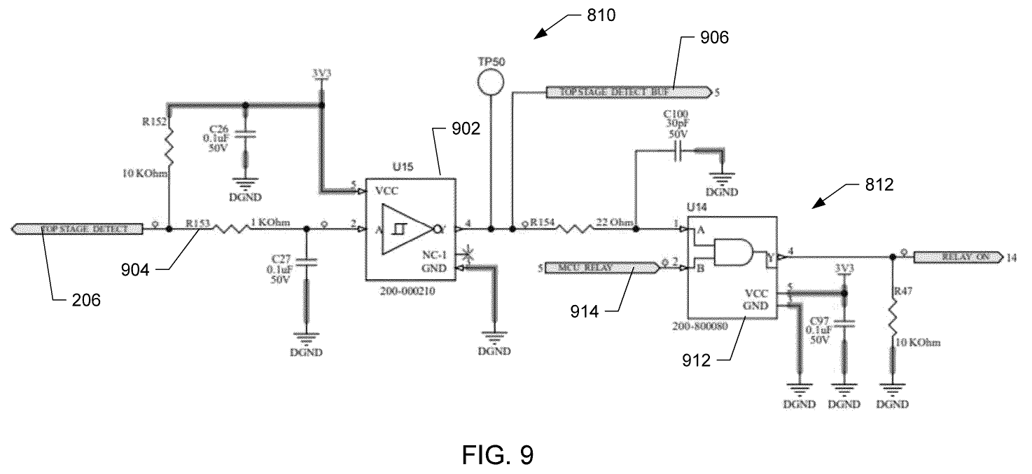

FIG. 9 is a diagram of the detection circuit 810 of FIG. 8, according to an example embodiment of the present disclosure. The example detection circuit 810 includes an inverter 902 with a Schmitt trigger, which receives a 3.3 DC voltage from the voltage regulator 708b. The detection circuit 810 also includes resistors R152, R153 and capacitors C26, C27 that regulate a voltage on an input line 904 to the inverter 902. When another docking apparatus 106 or the connectivity stage 104 is not connected, the inverter 902 is configured to provide a low signal or voltage to a detection buffer pin 906 of the microcontroller 802. However, when another docking apparatus 106 or the connectivity stage 104 is connected, the pin 206 and the input line 904 is pulled to ground or a lower voltage as a result of the pin 306 of the bottom data connector 304 being connected to ground or a lower voltage via, for example, a general-purpose input/output (“GPIO”) pin of a microcontroller. When this connection occurs, the inverter 902 is configured to output a higher voltage to the detection buffer pin 906 of the microcontroller 802, which indicates the connection of the other, higher docking apparatus 106 or the connectivity stage 104.

After detecting the connection of the other docking apparatus 106 or the connectivity stage 104, the microcontroller 802 is configured to cause or otherwise control the relay switch circuit 704 to switch or actuate to the closed state to enable the AC voltage to reach the AC outlet connector 202. As shown in FIG. 8, the microcontroller 802 may operate with a logic circuit 812 to control the relay switch circuit 704, which includes a relay driver 814 and a relay switch 816.

FIG. 9 also shows a circuit diagram of the logic circuit 812, according to an example embodiment of the present disclosure. The logic circuit 812 includes an AND gate 912 with a first input connected to an output of the inverter 902. A second input of the AND gate 912 is connected to an activation signal pin 914 of the microcontroller 802. An output of the AND gate 912 is connected to the relay driver 814 of the relay switch circuit 704. The AND gate 912 is configured to output a relay activation signal (e.g., a high voltage) when the output of the inverter 902 is high and the activation signal pin 914 includes a high voltage from the microcontroller 802. The high voltage may be 3.3 volts, 2.5 volts, 2.2 volts, for example. When either of the inputs to the AND gate 912 go to ground or a low voltage, the AND gate 912 is configured to output a low voltage or pull the input of the relay driver 814 to ground.

The example logic circuit 812 includes the AND gate 912 to provide fault-tolerant detection for detecting the connection with another docking apparatus 106 or the connectivity stage 104. This fault-tolerance prevents an AC voltage from being provided to the AC outlet connector 202 inadvertently. The relay switch 816 is only actuated to the closed position when the output of the inverter 902 is high, which only occurs when the pin 306 of the bottom data connector 304 pulls the pin 206 and the input line 904 to ground or to a low voltage, and when the microcontroller 802 provides a high voltage on the activation signal pin 914. Otherwise, the relay switch 816 is actuated to the open state or position. As such, static and other EMC artifacts cannot accidently trigger the relay switch 816.

It should be appreciated that the detection circuit 810 and the logic circuit 812 of FIG. 9 are only examples of circuits for detecting a connection and controlling the relay switch 816. In other examples, the detection circuit 810 and/or the logic circuit 812 may be integrated within circuitry of the microcontroller 802. Alternatively, the detection circuit 810 may include transistors, diodes, or other integrated circuit components to detect when another docking apparatus or the connectivity stage 104 is connected. In yet alternative embodiments, detection is made through a communication handshake with the other docking apparatus or the connectivity stage 104.

In some embodiments, the microcontroller 802 is configured to operate an algorithm specified by the instructions 806 for further detecting the connection of the other docking apparatus 106 or the connectivity stage 104. The algorithm may specify a time and/or voltage threshold for detecting the connection. For example, the algorithm may specify that the voltage received on the detection buffer pin 906 of the microcontroller 802 has to be high for at least 500 milliseconds, 1 second, 2 seconds, 5 seconds, etc. before a detection is detected. Additionally or alternatively, another threshold may specify a voltage limit, such as 3.0 volts. Detection of the connection may only occur if the signal output by the inverter 902 is greater than this voltage limit.

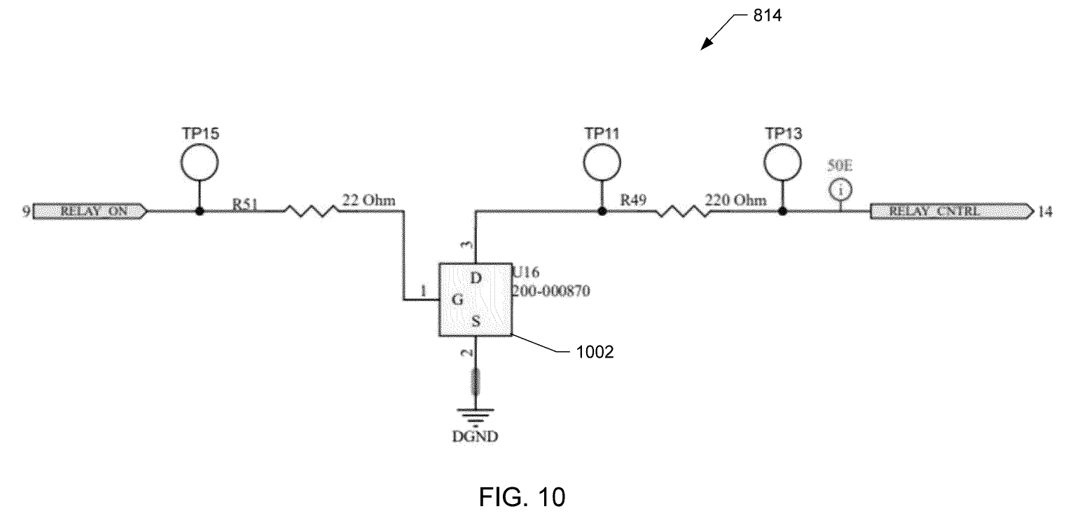

FIG. 10 is a diagram of the relay driver 814, according to an example embodiment of the present disclosure. The relay driver 814 includes an integrated circuit 1002 that is configured to output a relay control signal and/or an excitation voltage to the relay switch 816 in response to receive the relay activation signal from the logic circuit 812. The excitation voltage is sufficient to induce a magnetic field within coils of the relay 816 or power an actuator of a switch to move to a closed state. In some embodiments, the excitation voltage is 16 volts.

The relay switch 816 includes any relay switch that is configured to actuate from an open state to a closed state in response to receiving an excitation voltage. The relay switch 816 may include separate contacts for each of the power line, the earth-ground line, and the neutral line of the power bus 702. In other embodiments, the relay switch 816 may be replaced by an electronic switch, such as a power transistor.

Returning to FIG. 8, the microcontroller 802, the detection circuit 810, the logic circuit 812, and the relay driver 814 may be mounted on the PCB 710 of FIG. 7. The relay switch 816 may be mounted internally with the housing 130 and electrically connected to the relay driver 814. In other embodiments, the relay switch 816 may also be mounted to the PCB 710.

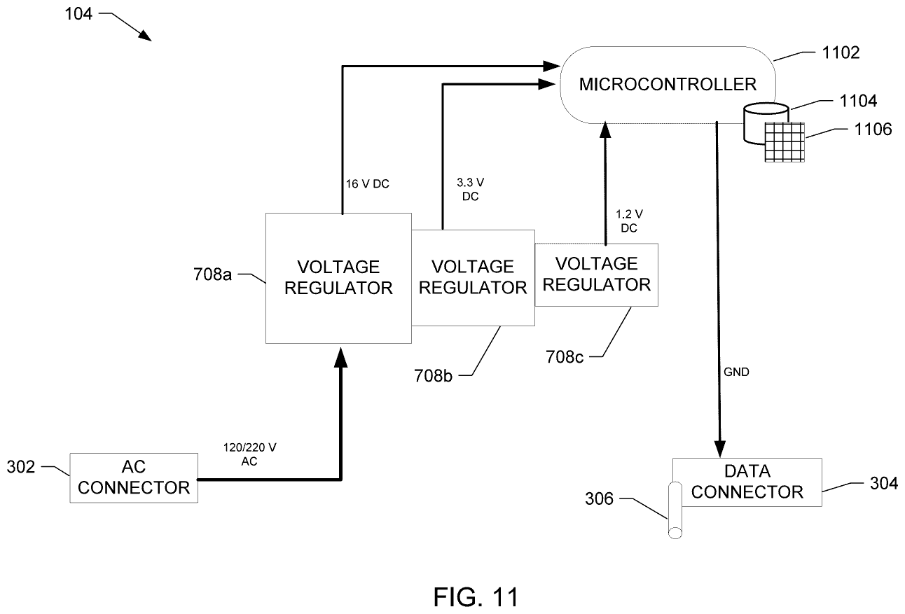

FIG. 11 is a diagram of the connectivity stage 104, according to an example embodiment of the present disclosure. Similar to the docking apparatus 106, the connectivity stage 104 includes the AC inlet connector 302 and the bottom data connector 304. The AC inlet connector 302 is configured to receive power from an AC outlet connector 202 of a lower connected docking apparatus 106. Similarly, the bottom data connector 304 is configured to connect to the top data connector 204 of a lower connected docking apparatus 106. As shown, a pin of a microcontroller 1102 of the connectivity stage 104 is pulled to ground or provided with a low voltage, which is electrically connected to the pin 306 of the bottom data connector 304. When the connectivity stage 104 is connected to a lower docking apparatus 106, the pin 306 connects to pin 206 of the top data connector 204, which causes the input to the inverter 902 of the detection circuit 810 to be pulled to the low voltage of ground.

The example microcontroller 1102 may include a memory 1104 that stores instructions 1106, which when executed by the microcontroller 1102, cause the microcontroller 1102 to perform the operations disclosed herein. The example microcontroller 1102 may include or be communicatively coupled to an Ethernet transceiver and a CAN interface. As discussed above, the microcontroller 1102 converts CAN messages from the docking apparatus 106 and/or connected medical devices to Ethernet messages for transmission to the gateway server 602. The microcontroller 1102 also converts Ethernet messages from the gateway server 602 to CAN messages for transmission to the docking apparatus 106 and/or connected medical devices. The example microcontroller 1102 also uses CAN messages for identifying positions of the docking apparatuses 106 within the hub connectivity station 100 and identifying positions of medical devices within the respective docking apparatus.

### Power Control Embodiment

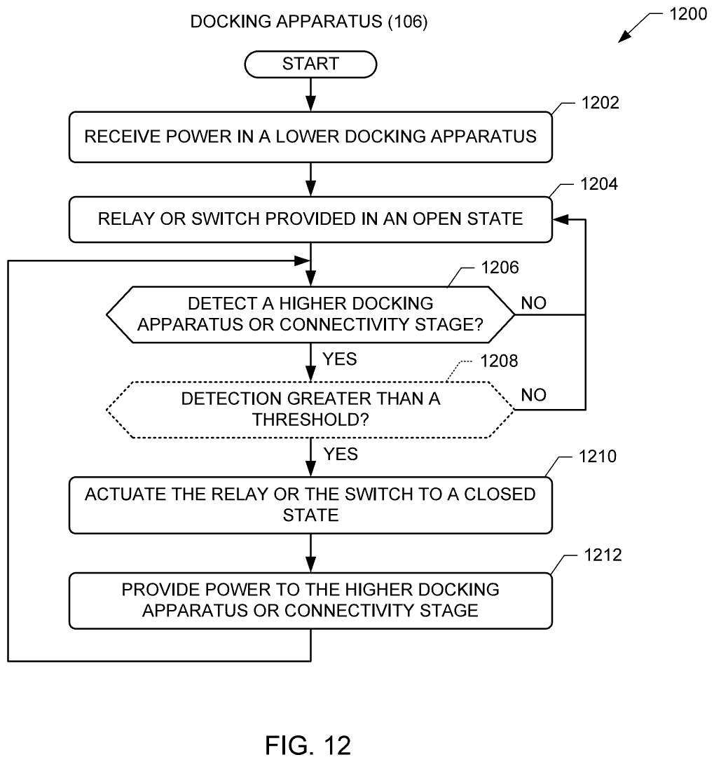

FIG. 12 shows a flow diagram illustrating an example procedure 1200 for controlling power between the docking apparatuses 106 and the connectivity stage 104 of the hub connectivity station 100, according to an example embodiment of the present disclosure. The example procedure 1200 may be carried out by, for example, the docking apparatus 106 described in conjunction with FIGS. 1 to 11. Although the procedure 1200 is described with reference to the flow diagram illustrated in FIG. 12, it should be appreciated that many other methods of performing the functions associated with the procedure 1200 may be used. For example, the order of many of the blocks may be changed, certain blocks may be combined with other blocks, and many of the blocks described are optional.

The procedure 1200 begins when power is provided to a lowest docking apparatus 106, such as the docking apparatus 106b or 106f of FIG. 4 (block 1202). Power may be provided when a power cord is connected to a lowest lower docking apparatus 106b and a power source. In this example, a clinician may create a stack for the hub connectivity station 100 by first powering the lowest docking apparatus and subsequently adding higher docking apparatuses 106, the connectivity stage 104, and the handle stage 102. The clinician may form the hub connectivity station 100 in this manner to prevent having to bend over or crouch down to connect a power cord to the bottom of the lowest docking apparatus, especially when six docking apparatuses 106 are stacked together.

After power is provided to the lowest docking apparatus 106, the microcontroller 802 in conjunction with the logic circuit 812 is configured to place the relay switch into an open state or position (block 1204). In some instances, the relay switch 816 is configured to default to the open state or position unless an excitation voltage is received. Next, the microcontroller 802 in conjunction with the detection circuit 810 determine whether a higher docking apparatus or the connectivity stage 104 has been connected (block 1206). As discussed above, the detection is made based on whether the pin 206 of the top data connector 204 is pulled to ground or a low voltage due to the corresponding pin 306 of the bottom data connector 306 of the higher docking apparatus or the connectivity stage 104 being pulled to ground or a low voltage. In alternative embodiments, the detection is made by detecting a high voltage applied to the pin 206 when the pin 306 of the bottom data connector 306 of the higher docking apparatus or the connectivity stage 104 is connected to a relatively higher voltage source.

When a connection is not detected, the procedure 1200 returns to block 1204 where the relay switch 816 is kept in the open state. However, when the connection of the higher docking apparatus or the connectivity stage 104 is detected, the microcontroller 802 may compare the detection signal to one or more thresholds, such as a time and/or voltage threshold (block 1208). The thresholds may prevent false detections due to electrical transient signals or electromagnetic interference. In an example, the detection needs to occur for a continuous time that meets or exceeds a time threshold. Additionally or alternatively, the output of the inverter 902 needs to meet and/or exceed a certain voltage to make a connection detection. In some embodiments, the comparison to one or more thresholds is omitted.

When at least one threshold is used and not satisfied, the procedure 1200 returns to block 1204 where the relay switch 816 is kept in the open state. However, when the at least one threshold is satisfied, the microcontroller 802 in conjunction with the logic circuit 812 and the relay driver 814 cause the relay switch 816 to actuate from the open state to a closed state or position (block 1210). The docking apparatus 106 then provides power to the AC outlet connector 202, thereby providing power to the higher docking apparatus or the connectivity stage 104 (block 1212). The example procedure 1200 then returns to checking whether the higher docking apparatus is still connected. The example procedure 1200 continues until power is removed from the lowest docking apparatus. Further, as additional docking apparatuses are added, each docking apparatus performs the procedure 1200.

It should be appreciated that the example procedure 1200 can be reversed. For example, the handle stage 102 and the connectivity stage 104 may first be connected together. Then, one or more docking apparatuses 106 are connected such that the stack of the hub connectivity station 100 is built downward. During this time, no power is connected. When the last docking apparatus 106 is connected, a power cord is connected, thereby providing power to the connectivity stage 104 and the docking apparatuses 106. However, the detection circuit 810 of the docking apparatuses 106 ensures that the AC outlet connector 202 never has a live voltage in the event the docking apparatuses 106 are disconnected when the power cord is still connected to the lowest docking apparatus 106.

### CONCLUSION

It will be appreciated that all of the disclosed methods and procedures described herein can be implemented using one or more computer programs or components. These components may be provided as a series of computer instructions on any conventional computer-readable medium, including RAM, ROM, flash memory, magnetic or optical disks, optical memory, or other storage media. The instructions may be configured to be executed by a processor, which when executing the series of computer instructions performs or facilitates the performance of all or part of the disclosed methods and procedures.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims.

It should be appreciated that 35 U.S.C. 112 (f) or pre-AIA 35 U.S.C 112, paragraph 6 is not intended to be invoked unless the terms “means” or “step” are explicitly recited in the claims. Accordingly, the claims are not meant to be limited to the corresponding structure, material, or actions described in the specification or equivalents thereof.

1. An infusion pump docking apparatus of a hub connectivity station, the apparatus comprising:
a housing;
an AC inlet connector positioned on a bottom side of the housing;
an AC outlet connector positioned on a top side of the housing and aligned with and electrically coupled to the AC inlet connector;
a relay switch circuit electrically connected between the AC outlet connector and the AC inlet connector;
a voltage regulator configured to convert an AC voltage received from the AC inlet connector to at least one DC voltage;
a microcontroller configured to receive at least one DC voltage from the voltage regulator;
a first data connector positioned on the bottom side of the housing;
a second data connector positioned on the top side of the housing and aligned with the first data connector; and
a detection circuit electrically connected between the microcontroller and the second data connector, the detection circuit configured to detect when a first data connector of another infusion pump docking apparatus is coupled to the infusion pump docking apparatus, the detection circuit configured to provide a signal to the microcontroller indicative of the connection of the other infusion pump docking apparatus,
wherein the microcontroller is configured to cause the relay switch circuit to close to enable the AC voltage to reach the AC outlet connector for powering the other infusion pump docking apparatus when connected to the infusion pump docking apparatus.

2. The apparatus of claim 1, further comprising:
a first pump connector configured to provide at least one DC voltage from the voltage regulator to a first infusion pump;
a first shelf and latch to mechanically connect to and support the first infusion pump;
a second pump connector configured to provide at least one DC voltage from the voltage regulator to a second infusion pump; and
a second shelf and latch to mechanically connect to and support the second infusion pump.

3. The apparatus of claim 1, wherein the other infusion pump docking apparatus is identical to the infusion pump docking apparatus.

4. The apparatus of claim 1, wherein the other infusion pump docking apparatus is a top-stage connectivity stage that omits an AC outlet connector, a relay switch circuit, a second data connector, and a detection circuit.

5. The apparatus of claim 1, wherein a pin of the first data connector is electrically coupled to ground or a low voltage,
wherein a pin of the second data connector is configured to electrically connect to the pin of the first data connector of the other infusion pump docking apparatus when connected, and
wherein the detection circuit is configured to detect the connection of the other infusion pump docking apparatus when the pin of the second data connector is pulled to the ground or the low voltage.

6. The apparatus of claim 5, wherein each of the first data connector and the second connector additionally includes pins for at least one of controller area network (“CAN”) communication, Ethernet communication, and universal asynchronous receiver-transmitter (“UART”) communication.

7. The apparatus of claim 5, wherein the detection circuit includes an inverter with a Schmitt trigger for detecting when the pin of the second data connector is pulled to the ground or the low voltage, an output of the inverter being electrically connected to the microcontroller.

8. The apparatus of claim 7, wherein the relay switch circuit includes:
a relay switch;
a logic circuit including a first input that is connected to the output from the inverter, a second input that is connected to an activation signal pin of the microcontroller, and an output for providing a relay activation signal; and
a relay driver configured to receive the relay activation signal and provide an excitation voltage to the relay switch, causing the relay switch or actuate to a closed position.

9. The apparatus of claim 7, wherein the microcontroller includes instructions stored in a memory that define an algorithm that when executed, cause the microcontroller to:
receive an output signal from the inverter;
detect the connection of the other infusion pump docking apparatus by comparing the output signal to a threshold; and
transmit an activation signal via an activation signal pin when the output signal exceeds the threshold.

10. The apparatus of claim 7, wherein the threshold includes at least one of a voltage amplitude value and a time value.

11. The apparatus of claim 1, wherein the detection circuit is included within a same circuit packaging as the microcontroller or integrally formed with the microcontroller.

12. An infusion pump docking apparatus of a hub connectivity station, the apparatus comprising:
an AC inlet connector positioned on a bottom side of a housing;
an AC outlet connector positioned on a top side of the housing and aligned with and electrically coupled to the AC inlet connector;
a relay switch circuit electrically connected between the AC outlet connector and the AC inlet connector;
a first data connector positioned on the bottom side of the housing;
a second data connector positioned on the top side of the housing and aligned with the first data connector;
a detection circuit electrically connected to the second data connector, the detection circuit configured to detect when a first data connector of another infusion pump docking apparatus is coupled to the infusion pump docking apparatus, the detection circuit configured to provide a signal indicative of the connection of the other infusion pump docking apparatus; and
a microcontroller electrically connected to the detection circuit, the microcontroller configured to:
receive the signal from the detection circuit, and
cause the relay switch circuit to close to enable AC voltage to reach the AC outlet connector for powering the other infusion pump docking apparatus when connected to the infusion pump docking apparatus.

13. The apparatus of claim 12, wherein the other infusion pump docking apparatus is identical to the infusion pump docking apparatus.

14. The apparatus of claim 12, wherein the other infusion pump docking apparatus is a top-stage connectivity stage that omits an AC outlet connector, a relay switch circuit, a second data connector, and a detection circuit.

15. The apparatus of claim 12, wherein a pin of the first data connector is electrically coupled to ground or a low voltage,
wherein a pin of the second data connector is configured to electrically connect to the pin of the first data connector of the other infusion pump docking apparatus when connected, and
wherein the detection circuit is configured to detect the connection of the other infusion pump docking apparatus when the pin of the second data connector is pulled to the ground or the low voltage.

16. The apparatus of claim 15, wherein each of the first data connector and the second connector additionally includes pins for at least one of controller area network (“CAN”) communication, Ethernet communication, and universal asynchronous receiver-transmitter (“UART”) communication.

17. The apparatus of claim 15, wherein the detection circuit includes an inverter with a Schmitt trigger for detecting when the pin of the second data connector is pulled to the ground or the low voltage, an output of the inverter being electrically connected to the microcontroller.

18. The apparatus of claim 17, wherein the relay switch circuit includes:
a relay switch;
a logic circuit including a first input that is connected to the output from the inverter, a second input that is connected to an activation signal pin of the microcontroller, and an output for providing a relay activation signal; and
a relay driver configured to receive the relay activation signal and provide an excitation voltage to the relay switch, causing the relay switch or actuate to a closed position.

19. The apparatus of claim 17, wherein the microcontroller includes instructions stored in a memory that define an algorithm that when executed, cause the microcontroller to:
receive an output signal from the inverter;
detect the connection of the other infusion pump docking apparatus by comparing the output signal to a threshold; and
transmit an activation signal via an activation signal pin when the output signal exceeds the threshold.

20. The apparatus of claim 19, wherein the threshold includes at least one of a voltage amplitude value and a time value.
